SEQUENCE LISTING (4 sequences)

SEQ ID NO: 1 - protein, 19 aa, synthetic construct
MEFGLSWVFLVALFRGVQC

SEQ ID NO: 2 - protein, 10 aa, synthetic construct
EQKLISEEDL

SEQ ID NO: 3 - protein, 49 aa, synthetic construct
AVGQDTQEVIVVPHSLPFKVVVISAILALVVLTIISLIILIMLWQKKPR

SEQ ID NO: 4 - DNA, 1254 bp, synthetic construct
  source 1..1254: mol_type = other DNA
tctgataacggaccccaaaatcagcgcaacgccccgcggatcactttcggcggaccaagcgacagcaccgggtctaaccagaatggcgagaggagcggggctcgcagcaaacagcgtagaccccaaggactgccaaataacaccgctagttggttcacagccttgacccagcacggaaaggaggatctgaaattcccaagagggcagggcgtcccaatcaacactaatagttctcctgatgaccagataggttattacaggagggccacccggcggatccgcggcggggatggaaagatgaaagatttaagtccacgatggtatttttactatctgggaactggccctgaagcagggttgccgtacggggctaacaaggacggtattatttgggttgctacagaaggggcgctgaatactcctaaagaccacattgggacccgtaatcctgctaataacgccgccatcgtgttgcagctgcctcaaggcactacccttcccaaaggattctatgctgagggttcacggggcggctctcaggcatcctcaaggtccagtagcagatcaaggaacagctcccgcaattccactccaggctcatctaggggtactagccctgcacggatggctgggaatgggggggatgcagcactggcgctcttattgctcgatcgcctcaatcagttagagtcaaagatgagtgggaaaggacagcagcagcaggggcagacggtgaccaaaaagtccgcagctgaggcgagcaagaaacccaggcagaagcggacagcgaccaaggcttacaatgtgacccaagccttcggacggcgcggtccagagcagacccagggcaacttcggggatcaggagcttattcgtcaaggcactgattataagcactggccccagatcgcacagtttgcccccagtgcctccgctttttttgggatgagcagaattggcatggaggtgacaccttcaggcacgtggctcacatacaccggggcaattaagctggatgataaggatcccaatttcaaggaccaggtcattcttctgaacaagcacattgatgcctacaagacattcccacccaccgagcccaagaaggacaagaagaagaaagcagatgagacacaagcccttccacaacggcagaaaaaacaacagactgttacgctgctgcccgctgccgacctggacgacttttctaagcaattgcagcagagtatgtcatccgcagactcaactcaagcc

(19) United States
(12) Patent Application Publication
(10) Pub. No.: US 2024/0271124 A1
(43) Pub. Date: Aug. 15, 2024

(54) Cell Surface Expressed Protein Variants, Uses, and Composition Related Thereto

(71) Applicant: Emory University, Atlanta, GA (US)

(72) Inventors: Eric Ortlund, Decatur, GA (US); Filipp Frank, Decatur, GA (US); William Hudson, Spring, TX (US)

(21) Appl. No.: 18/439,733

(22) Filed: Feb. 12, 2024

Related U.S. Application Data
(60) Provisional application No. 63/445,438, filed on Feb. 14, 2023.

(51) Int. Cl. C12N 15/10; C07K 14/005

(52) U.S. Cl. CPC C12N 15/1065; C07K 14/005; C12N 15/1037; C12N 2770/20022

(57) ABSTRACT

This disclosure relates to libraries of recombinant protein variants expressed on the surface of cells from expression constructs and uses in methods analyzing the impact of one or more mutations on affinity binding to specific binding agents. In certain embodiments, this disclosure relates to methods of contacting binding agents used in diagnostic test with libraries of cell surface expressed protein variants to analyze binding affinity. In certain embodiments, the protein variants are expressed on the exterior of cells containing intracellular nucleic acids with barcodes that correlate to specific amino acid variants sequences.

### CROSS-REFERENCE TO RELATED APPLICATIONS

This application claims the benefit of U.S. Provisional Application No. 63/445,438 filed Feb. 14, 2023. The entirety of this application is hereby incorporated by reference for all purposes.

### STATEMENT REGARDING FEDERALLY SPONSORED RESEARCH OR DEVELOPMENT

This invention was made with government support under EB027690 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22101US.xml. The XML file is 6 KB, was created on Feb. 9, 2024, and is being submitted electronically via the USPTO patent electronic filing system.

### BACKGROUND

Widespread and frequent testing is critical to prevent the spread of COVID-19, and rapid antigen tests are the diagnostic tool of choice in many settings. Most rapid antigen tests detect the presence of the SARS-CoV-2 nucleocapsid (N) protein due to its high abundance in virions and infected individuals. The N protein is involved in multiple steps in the viral life cycle, playing important roles in viral RNA replication and packaging. With new viral variants continuously emerging and spreading rapidly, the effect of variant mutations on antigen test performance is a major concern. Thus, there is a need to identify methods for monitoring the ability of antibodies in test kits to sufficiently bind SARS-CoV-2 nucleocapsid (N) protein mutations that might jeopardize accuracy.

Craig et al. report identification of genetic variants using bar-coded multiplexed sequencing. Nat Methods, 2008, 5(10):887-93.

Fowler et al. report measuring the activity of protein variants on a large scale using deep mutational scanning. Nature protocols, 2014, 9(9): 2267-2284.

Starr et al. report deep mutational scanning of the SARS-CoV-2 receptor binding domain reveals constraints on folding and ACE2 binding. Cell 182, 1295-1310, 2020.

Chan et al. report engineering human ACE2 to optimize binding to the spike protein of SARS-CoV-2. Science, 2020, 369, 1261-1265.

Greaney et al. report comprehensive mapping of mutations in the SARS-CoV-2 receptor-binding domain that affect recognition by polyclonal human plasma antibodies. Cell Host & Microbe, 2021, 29, 463-476.

References cited herein are not an admission of prior art.

### SUMMARY

This disclosure relates to libraries of recombinant protein variants expressed on the surface of cells from expression constructs and uses in methods of analyzing the impact of one or more mutations on affinity binding to specific binding agents. In certain embodiments, this disclosure relates to methods of contacting binding agents used in diagnostic test with libraries of cell surface expressed protein variants to analyze binding affinity. In certain embodiments, the protein variants are expressed on the exterior of cells containing intracellular nucleic acids with barcodes that correlate to specific amino acid variants sequences.

In certain embodiments, the libraries are used in methods to confirm the effectiveness or non-effectiveness of the test binding agent to identify circulating mutant microbial strains, e.g., viral strains, coronaviral strains of SARS-CoV-2, coronavirus nucleocapsid sequences, coronavirus spike protein sequences, influenza proteins, or influenza virus nucleoproteins or capsid proteins.

In certain embodiments, this disclosure relates to libraries of coronavirus nucleocapsid expression constructs comprising: nucleic acids having barcode sequences and segments encoding peptides; the peptides comprising an N-terminal signal peptide sequence for translocation of the peptides across a cell membrane; a coronavirus nucleocapsid sequence; and a transmembrane domain sequence for insertion into a cell membrane C-terminal to the coronavirus nucleocapsid sequence; wherein each of the encoded peptides are unique coronavirus nucleocapsid sequences with at least a single amino acid variant when compared to a base coronavirus nucleocapsid sequence; and wherein the encoded peptides with the amino acid variant sequences are correlated to single unique nucleotide barcode sequences.

In certain embodiments, the encoded peptides include single variants at each position in the base coronavirus nucleocapsid sequence, wherein the single amino acid variants are alanine, arginine, asparagine, aspartic acid, cysteine, glutamine, glutamic acid, glycine, histidine, isoleucine, leucine, lysine, phenylalanine, proline, serine, threonine, tryptophan, tyrosine, methionine, and valine at each position within the base coronavirus nucleocapsid sequence.

In certain embodiments, nucleic acids encoding the coronavirus nucleocapsid sequence comprises the codon optimized base sequence (SEQ ID NO: 4)

TCTGATAACGGACCCCAAAATCAGCGCAACGCCCCGCGGATCACTTTCGGCG GACCAAGCGACAGCACCGGGTCTAACCAGAATGGCGAGAGGAGCGGGGCTCGCAG CAAACAGCGTAGACCCCAAGGACTGCCAAATAACACCGCTAGTTGGTTCACAGCCT TGACCCAGCACGGAAAGGAGGATCTGAAATTCCCAAGAGGGCAGGGCGTCCCAATC AACACTAATAGTTCTCCTGATGACCAGATAGGTTATTACAGGAGGGCCACCCGGCG GATCCGCGGGGGGATGGAAAGATGAAAGATTTAAGTCCACGATGGTATTTTTACT ATCTGGGAACTGGCCCTGAAGCAGGGTTGCCGTACGGGGCTAACAAGGACGGTATT ATTTGGGTTGCTACAGAAGGGGCGCTGAATACTCCTAAAGACCACATTGGGACCCGT AATCCTGCTAATAACGCCGCCATCGTGTTGCAGCTGCCTCAAGGCACTACCCTTCCC AAAGGATTCTATGCTGAGGGTTCACGGGGCGGCTCTCAGGCATCCTCAAGGTCCAGT AGCAGATCAAGGAACAGCTCCCGCAATTCCACTCCAGGCTCATCTAGGGGTACTAG CCCTGCACGGATGGCTGGGAATGGGGGGGATGCAGCACTGGCGCTCTTATTGCTCG ATCGCCTCAATCAGTTAGAGTCAAAGATGAGTGGGAAAGGACAGCAGCAGCAGGGG CAGACGGTGACCAAAAAGTCCGCAGCTGAGGCGAGCAAGAAACCCAGGCAGAAGC GGACAGCGACCAAGGCTTACAATGTGACCCAAGCCTTCGGACGGCGCGGTCCAGAG CAGACCCAGGGCAACTTCGGGGATCAGGAGCTTATTCGTCAAGGCACTGATTATAA GCACTGGCCCCAGATCGCACAGTTTGCCCCCAGTGCCTCCGCTTTTTTTGGGATGAG CAGAATTGGCATGGAGGTGACACCTTCAGGCACGTGGCTCACATACACCGGGGCAA TTAAGCTGGATGATAAGGATCCCAATTTCAAGGACCAGGTCATTCTTCTGAACAAGC ACATTGATGCCTACAAGACATTCCCACCCACCGAGCCCAAGAAGGACAAGAAGAAG AAAGCAGATGAGACACAAGCCCTTCCACAACGGCAGAAAAAACAACAGACTGTTAC GCTGCTGCCCGCTGCCGACCTGGACGACTTTTCTAAGCAATTGCAGCAGAGTATGTC ATCCGCAGACTCAACTCAAGCC wherein a codon mutation/variant results in a single amino acid change.

In certain embodiments, the codon mutation/variant for alanine is GCC, the codon mutation/variant for cysteine is TGC, the codon mutation/variant for aspartic acid is GAC, the codon mutation/variant for glutamic acid is GAG, the codon mutation/variant for phenylalanine is TTC, the codon mutation/variant for glycine is GGC, the codon mutation/variant for histidine is CAC, the codon mutation/variant for isoleucine is ATC, the codon mutation/variant for lysine is AAG, the codon mutation/variant for leucine is CTG, the codon mutation/variant for methionine of is ATG, the codon mutation/variant for asparagine of is AAC, the codon mutation/variant for proline of is CCC, the codon mutation/variant for glutamine is CAG, the codon mutation/variant for arginine is AGA, the codon mutation/variant for threonine is ACC, the codon mutation/variant for valine is GTG, the codon mutation/variant for tryptophan is TGG, and the codon mutation/variant for tyrosine is TAC.

In certain embodiments, the N-terminal signal peptide sequence for translocation of the peptides across a cell membrane comprises (SEQ ID NO: 1) MEFGLSWVFLVALFRGVQC.

In certain embodiments, the transmembrane domain sequence for insertion into a cell membrane comprises (SEQ ID NO: 3) AVGQDTQEVIVVPHSLPFKVVVISAILALVVLTIIS LIILIMLWQKKPR.

In certain embodiments, a peptide label sequence is between the signal peptide and the coronavirus nucleocapsid sequence. In certain embodiments, the peptide label sequence comprises (SEQ ID NO: 2) EQKLISEEDL.

In certain embodiments, this disclosure relates to a library of cells expressing coronavirus nucleocapsid variant expression constructs as disclosed herein.

In certain embodiments, this disclosure relates to a non-transitory computer readable medium comprising the data of each of the unique nucleotide barcode sequences that are associated with each coronavirus nucleocapsid sequence variants as reported herein. In certain embodiments, the non-transitory computer readable medium is on a memory storage device, a desktop or portable computer, hard drive, portable drive, workstation, server, cloud server, or any other computer system.

In certain embodiments, this disclosure relates to methods of detecting a test binding agent for binding to coronavirus nucleocapsid variants comprising: contacting a test binding agent specific for a coronavirus nucleocapsid with a library of cells expressing coronavirus nucleocapsid variant expression constructs as disclosed herein providing cells with the test binding agent bound to coronavirus nucleocapsid variants on the cells; contacting the test binding agent bound to coronavirus nucleocapsid variant on the cells with a secondary labeling agent capable of binding the test binding agent bound to the coronavirus nucleocapsid variants on the cells providing secondary labeling agent bound to the coronavirus nucleocapsid variants on the cells.

In certain embodiments, methods further comprise separating cells with the secondary labeling agent bound to the coronavirus nucleocapsid variant.

In certain embodiments, methods further comprise sequencing the barcodes of the separated cells and associating the barcodes to a specific coronavirus nucleocapsid variant. In certain embodiments, methods further comprise associating the barcodes to a specific coronavirus nucleocapsid variant is by comparing the barcode sequence to sequences on non-transitory computer readable medium comprising the data of each of the unique nucleotide barcode sequence that are associated with each of coronavirus nucleocapsid sequence variants in the library.

In certain embodiments, methods further comprise quantifying the binding affinity of the test binding agent associated to the coronavirus nucleocapsid variants on the cells.

In certain embodiments, methods further comprise contacting the cells with a labeling agent capable of binding a peptide label sequence between the signal peptide and the coronavirus nucleocapsid sequence and detecting the labeling agent indicating that the test compound is bound to the coronavirus nucleocapsid sequence.

### BRIEF DESCRIPTION OF THE SEVERAL VIEWS OF THE DRAWINGS

FIG. 1 illustrates a SARS-CoV-2 nucleocapsid mammalian surface-display platform construct design for mammalian surface-display and schematic for detection of surface-displayed nucleocapsid protein. A signal peptide and Myc-tag were introduced at the N terminus and a transmembrane helix at the C terminus of the nucleocapsid protein. The construct was cloned into a lentiviral expression plasmid containing a GFP marker expressed from the same mRNA via an internal ribosomal entry site (IRES). Flow cytometry analysis of HEK293 cells stably expressing surface-displayed nucleocapsid indicates the majority of cells are GFP+ and Myc+ (>90%). GFP+Myc+-gated cells were analyzed for anti-N antibody binding signal (via phycoerythrin [PE]-labeled secondary antibody). Titration experiments for antibodies used provide normalized median fluorescence intensity (MFI) signal for PE. Validation of dissociation constants were determined by mammalian display with dissociation constants from biolayer interferometry (BLI) with recombinant protein.

FIG. 2 illustrates a barcode deep mutational scanning approach for determining escape mutations of nucleocapsid (N) specific antibodies. Fifteen (15) nucleotides for each of the barcodes were added to a site-saturation library containing point mutations in the nucleocapsid protein sequence, and the resulting constructs were cloned into a lentiviral expression plasmid (pLVX-IRES-ZsGreen). PacBio™ long-read sequencing was employed to associate unique barcodes with amino acid variants. The library was transduced into mammalian cells (HEK293), such that each cell expresses a single nucleocapsid variant.

FIGS. 3A-3C shows data on the performance of diagnostic antibodies and tests against variants of concern.

FIG. 3A shows variants and the associated mutations in samples used for laboratory testing. Circles mark mutations in the consensus sequence of a variant, which is not present in the remnant samples used for testing.

FIG. 3B shows normalized and weighted escape scores, EW, for mutations shown in (3A): EW=Ei,j×Etotal,j, where Ei,j is the normalized escape score of mutation I at position j (0<Ei,j<1), and Etotal,j is the normalized total escape score at position j (0<Etotal,j,<1).

FIG. 3C shows results of diagnostic tests with pools of sequence-verified remnant clinical samples. LODs are shown as ACT values compared with a reference sample: Wuhan WA1 when available; B.1.2 in all other cases (tests 2, 3, and 11). Omicron samples were collected at a later time and evaluated separately (checkmarks identify positive test results). All tests were able to detect the Omicron variant in remnant clinical samples. n.t., not tested; n.d., not detected (i.e., the test did not detect the virus, even at the highest virus concentration).

### DETAILED DISCUSSION

Before the present disclosure is described in greater detail, it is to be understood that this disclosure is not limited to embodiments described, and as such may, of course, vary. It is also to be understood that the terminology used herein is for describing particular embodiments only, and is not intended to be limiting, since the scope of the present disclosure will be limited only by the appended claims or as amended during prosecution.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. Further, the dates of publication provided could be different from the actual publication dates that may need to be independently confirmed.

As will be apparent to those of skill in the art that upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and infers that the example is not necessarily limited to the specific example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. patent law in that they are inclusive or open-ended and do not exclude additional, unrecited elements or method steps.

“Consisting essentially of” or “consists of” or the like, have the meaning ascribed to them in U.S. patent law in that when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

As used herein, the term “cell” refers to a biological compartment containing a lipid membrane and cytosol which may contain a nucleus containing genetic material, mitochondria, and other organelles.

As used herein, unless the context suggests otherwise, “separating” refers to purify the cells from other particles or impurities that do not contain or contain less of a target molecule on the surface. One method of selecting proteins that are on the outside of a cells is to provide a specific binding agent, such as a primary antibody, and further trap the primarily antibody bound to the cell using a secondary antibody that is conjugated to magnetic beads. The magnetic beads can be captured by a magnetic field and separated from the rest of a solution. In another method, secondary antibodies contain a fluorescent marker, and the cells can be separated using flow cytometry and/or fluorescence activated sorting.

One can coat or conjugate the cells with a fluorescent molecule to further “stain” the cells within a sample. As these cells passed through the stream, the laser light would excite the fluorescent core, tag, or fluorochrome to emit photons of light, e.g., at a higher wavelength (e.g., fluorescein isothiocyanate emits light at ˜530 nm when excited by a 488 nm laser). This light can be collected and used to further categorize the cells or particles in one or more a detectable fluorescent pattern(s) in addition to forward scatter and side scatter depending on the position of detectors of the fluorescent light or scattered light.

The term “fluorescence-activated sorting,” “fluorescence-activated cell sorting” or “FACS” refers to a method of sorting a mixture of cells into two or more areas, typically one cell at a time, based upon the fluorescent characteristics of each particle or cell. It is typically accomplished by applying an electrical charge and separating by movement through an electrostatic field. Typically, a vibrating mechanism causes a stream of cells to break into individual droplets. Just prior to droplet formation, a cell in a fluid pass through an area for measuring fluorescence. An electrical charging mechanism is configured at the point where the stream breaks into droplets. Based on the fluorescence intensity measurement, a respective electrical charge is imposed on the droplet as it breaks from the stream. The charged droplets then move through an electrostatic deflection system that diverts droplets into areas based upon their relative charge. In some systems, the charge is applied directly to the stream, and the droplet breaking off retains charge of the same sign as the stream. In other systems, a charge is provided on a conduit inducing an opposite charge on the droplet.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “specific binding agent” refers to a molecule, such as a proteinaceous molecule, that binds a target molecule with a greater affinity than other random molecules or proteins. Examples of specific binding agents include an antibody that bind an epitope of an antigen or a receptor which binds a ligand. In certain embodiments, “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule, i.e., an antigen to the antibody. The immune system of the animal will create an antibody to specifically bind the antigen, and thereby targeting the antigen for degradation or elimination, or any cell or organism attached to the antigen. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, the term “antibody” is intended to include natural antibodies, monoclonal antibody, or non-naturally produced synthetic antibodies, such as specific binding single chain antibodies, bispecific antibodies, or fragments thereof. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

From a structural standpoint, an antibody is a combination of proteins: two heavy chain proteins and two light chain proteins. The heavy chains are longer than the light chains. The two heavy chains typically have the same amino acid sequence. Similarly, the two light chains typically have the same amino acid sequence. Each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

As used herein, the term “ligand” refers to an organic molecule, i.e., substantially comprised of carbon, hydrogen, sulfur, and oxygen, that binds a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin may bind the glycan. As the glycan is typically smaller and surrounded by the lectin during binding, it may be considered a ligand even though it is a receptor of the lectin binding signal on the cell surface. In another example, a double stranded oligonucleotide sequence contains two complimentary nucleic acid sequences. Either of the single stranded sequences may be consider the ligand or receptor of the other. In certain embodiments, a ligand is contemplated to be a compound that has a molecular weight of less than 500 or 1,000. In certain embodiments, a receptor is contemplated to be a compound that has a molecular weight of greater than 2,000 or 5,000. In any of the embodiments disclosed herein the position of a ligand and a receptor may be switched.

Polypeptides can be produced by any commonly used method. Typical examples include the recombinant expression in suitable host systems, e.g., cell, mammalian cell, bacteria, or yeast. In general, the polypeptides may be produced by living host cells that have been genetically engineered to produce the polypeptide. Methods of genetically engineering cells to produce proteins are well known in the art. See e.g., Ausubel et al., eds. (1990), Current Protocols in Molecular Biology (Wiley, New York). Such methods include introducing nucleic acids that encode and allow expression of the polypeptide into host cells. These host cells can be bacterial cells, fungal cells, or animal cells grown in culture. In one embodiment, polypeptides are produced in mammalian cells. Typical mammalian host cells for expressing the peptide include Chinese Hamster Ovary (CHO cells), lymphocytic cell lines, e.g., NS0 myeloma cells, SP2 cells, COS cells.

In addition to the nucleic acid sequences encoding the peptide, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). For example, typically the selectable marker gene confers resistance to drugs, such as G418, hygromycin, or methotrexate, on a host cell into which the vector has been introduced.

Standard molecular biology techniques can be used to prepare the recombinant expression vector, transfect the host cells, select for transformants, culture the host cells and recover the peptides or cells coated with the peptide from the culture medium. For example, the peptides or cells can be isolated by affinity chromatography.

In certain embodiments, this disclosure relates to nucleotide sequences or nucleic acids that encode the peptides disclosed herein, genetic constructs that include the foregoing nucleotide sequences or nucleic acids and one or more elements for genetic constructs known per se. In certain embodiments, this disclosure relates to hosts or host cells that contain such nucleotide sequences or nucleic acids, and/or that express (or are capable of expressing), the peptides disclosed herein.

In certain embodiments, this disclosure relates to methods for preparing peptides or cells expressing the peptide constructs disclosed herein, which method comprises cultivating or maintaining a host cell as described herein under conditions such that said host cell produces or expresses the peptide constructs disclosed herein.

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular (somatic) or cell-free expression system. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a peptide disclosed herein in operable combination with a heterologous promoter.

A “label” refers to a detectable moiety that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous peptide in the receptor. A label includes the incorporation of a radiolabeled amino acid or the covalent attachment of biotinyl moieties to a peptide that can be detected by marked avidin (for example, streptavidin containing a fluorescent marker or enzymatic activity that can be detected by optical or colorimetric methods). Various methods of labeling peptides and glycoproteins are known in the art and may be used. Examples of labels for peptides include, but are not limited to, the following: radioisotopes or radionucleotides (such as ¹⁸F, ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined peptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms (linking groups) of various lengths to reduce potential steric hindrance.

In certain embodiments, the disclosure relates to recombinant peptides comprising sequences disclosed herein or variants or fusions thereof wherein interior amino acid sequence, the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a peptide disclosed herein. In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env. In certain embodiments, the recombinant vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating,” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general-purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that because some of the constituent system components and method steps may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

Contacting Binding Agents Used in Diagnostic Test with Libraries of Cell Surface Expressed Protein Variants to Analyze Binding Affinity

This disclosure relates to contacting binding agents used in diagnostic test with libraries of cell surface expressed protein variants to analyze binding affinity. In certain embodiments, the protein variants are expressed on the exterior of cells containing intracellular nucleic acids with barcodes that correlate to specific amino acid variants sequences encoded and expressed by vectors or other nucleic acids. In certain embodiments, the libraries are used in methods to confirm the effectiveness or non-effectiveness of the test binding agent to identify mutant microbial strains, e.g., viral, bacterial, fungal, or other microbial. In certain embodiments the viral microbial protein is a nucleocapsid sequence, spike protein sequence, or other viral capsid/coat protein.

In certain embodiments, the libraries are used in methods to confirm the effectiveness or non-effectiveness of the test binding agent to identify circulating mutant microbial strains, e.g., viral strains, coronaviral strains of SARS-CoV-2, coronavirus nucleocapsid sequence, coronavirus spike protein sequence influenza protein, or influenza virus nucleoprotein or capsid protein such as hemagglutinin (HA) and neuraminidase (NA).

SARS-Cov2 nucleocapsid protein is involved in multiple steps in the viral life cycle, playing important roles in viral RNA replication and packaging. It consists of two folded regions, i.e., the RNA-binding domain (N-RBD) and the dimerization domain (N-DD), surrounded by three disordered regions. Herein is describe a platform for mammalian surface-display of SARS-CoV-2 nucleocapsid, an intra-cellular protein, which allows for direct and quantitative measurement of antibody binding.

In certain embodiments, constructs and methods disclosed herein are used to evaluate detection reagents (e.g. monoclonal Abs, nanobodies, aptamers, affirmers) against a full length SARS-Cov2 nucleocapsid protein library containing substantially all possible point mutations over the entire protein sequence for mapping high-resolution native epitopes, both conformational and linear, capturing 3 dimensional epitopes wherein one can measure all possible mutations to enable residue-level epitope definition possible escape mutations (mutations that weaken binding to the detection reagent). In certain embodiments, it is contemplated that assay methods are efficient because many point mutations that may arise in the target sequence will be tested in single set of steps.

In certain embodiments, this disclosure relates to methods of screening a binding agent across a library of microbial protein variants and determining the binding affinity against each variant. In certain embodiments, this disclosure relates to a library of cell surface microbial protein expression constructs comprising nucleic acids having barcode sequences and sequences encoding peptides, wherein the peptides comprise an N-terminal signal peptide sequence for translocation of the peptide across a cell membrane, a microbial sequence, and a transmembrane domain sequence for insertion into a cell membrane which is on the C-terminal end of the microbial sequence; wherein each of the encoded peptides are the same sequences with only one or at least a single amino acid variant when compared to a base peptide sequence; and wherein the encoded peptides with the amino acid variants are correlated to single unique nucleotide barcode sequences for each cell, wherein barcode variants within a sample are used to identify/count the variants contained therein. In certain embodiments, the base peptide sequence is a predetermined consensus sequence, variant of interest, or variant of concern.

In certain embodiments, the protein variants are expressed on the exterior of cells containing intracellular nucleic acids and expressed by vectors or other nucleic acids with or without barcodes, i.e., vector sequencing where one can readout the data in a computer assisted manner though direct sequencing of the viral protein/nucleocapsid/spike variants.

In certain embodiments, the nucleic acids encoding the coronavirus nucleocapsid sequence comprises the base codon optimized nucleocapsid nucleotide sequence of (SEQ ID NO: 4)

TCTGATAACGGACCCCAAAATCAGCGCAACGCCCCGCGGATCACTTTCGGCGGACC AAGCGACAGCACCGGGTCTAACCAGAATGGCGAGAGGAGCGGGGCTCGCAGCAAA CAGCGTAGACCCCAAGGACTGCCAAATAACACCGCTAGTTGGTTCACAGCCTTGAC CCAGCACGGAAAGGAGGATCTGAAATTCCCAAGAGGGCAGGGCGTCCCAATCAACA CTAATAGTTCTCCTGATGACCAGATAGGTTATTACAGGAGGGCCACCCGGCGGATCC GCGGCGGGGATGGAAAGATGAAAGATTTAAGTCCACGATGGTATTTTTACTATCTGG GAACTGGCCCTGAAGCAGGGTTGCCGTACGGGGCTAACAAGGACGGTATTATTTGG GTTGCTACAGAAGGGGCGCTGAATACTCCTAAAGACCACATTGGGACCCGTAATCCT GCTAATAACGCCGCCATCGTGTTGCAGCTGCCTCAAGGCACTACCCTTCCCAAAGGA TTCTATGCTGAGGGTTCACGGGGCGGCTCTCAGGCATCCTCAAGGTCCAGTAGCAGA TCAAGGAACAGCTCCCGCAATTCCACTCCAGGCTCATCTAGGGGTACTAGCCCTGCA CGGATGGCTGGGAATGGGGGGGATGCAGCACTGGCGCTCTTATTGCTCGATCGCCTC AATCAGTTAGAGTCAAAGATGAGTGGGAAAGGACAGCAGCAGCAGGGGCAGACGG TGACCAAAAAGTCCGCAGCTGAGGCGAGCAAGAAACCCAGGCAGAAGCGGACAGC GACCAAGGCTTACAATGTGACCCAAGCCTTCGGACGGCGCGGTCCAGAGCAGACCC AGGGCAACTTCGGGGATCAGGAGCTTATTCGTCAAGGCACTGATTATAAGCACTGG CCCCAGATCGCACAGTTTGCCCCCAGTGCCTCCGCTTTTTTTGGGATGAGCAGAATT GGCATGGAGGTGACACCTTCAGGCACGTGGCTCACATACACCGGGGCAATTAAGCT GGATGATAAGGATCCCAATTTCAAGGACCAGGTCATTCTTCTGAACAAGCACATTGA TGCCTACAAGACATTCCCACCCACCGAGCCCAAGAAGGACAAGAAGAAGAAAGCA GATGAGACACAAGCCCTTCCACAACGGCAGAAAAAACAACAGACTGTTACGCTGCT GCCCGCTGCCGACCTGGACGACTTTTCTAAGCAATTGCAGCAGAGTATGTCATCCGC AGACTCAACTCAAGCC wherein codon mutation/variant substitution(s) results in a single amino acid change at each amino acid position.

In certain embodiments, the codon mutation for an alanine variant is GCC, the codon mutation for a cysteine variant is TGC, the codon mutation for an aspartic acid variant is GAC, the codon mutation for a glutamic acid variant is GAG, the codon mutation for a phenylalanine variant is TTC, the codon mutation for a glycine variant is GGC, the codon mutation for a histidine variant is CAC, the codon mutation for an isoleucine variant is ATC, the codon mutation for a lysine variant is AAG, the codon mutation for a leucine variant is CTG, the codon mutation for a methionine variant of is ATG, the codon mutation for an asparagine variant is AAC, the codon mutation for a proline variant is CCC, the codon mutation for a glutamine variant is CAG, the codon mutation for an arginine variant is AGA, the codon mutation for a threonine variant is ACC, the codon mutation for a valine variant is GTG, the codon mutation for a tryptophan variant is TGG, and the codon mutation for a tyrosine variant is TAC.

In certain embodiments, the N-terminal signal peptide sequence for translocation of the peptides across a cell membrane comprises (SEQ ID NO: 1) MEFGLSWVFLVALFRGVQC.

In certain embodiments, the transmembrane domain sequence for insertion into a cell membrane comprises (SEQ ID NO: 3) AVGQDTQEVIVVPHSLPFKVVVISAILALVVLTIIS LIILIMLWQKKPR.

In certain embodiments, a peptide label sequence is between the signal peptide and the coronavirus nucleocapsid sequence. In certain embodiments, the peptide label sequence comprises (SEQ ID NO: 2) EQKLISEEDL.

In certain embodiments, this disclosure relates to contacting binding agents used in diagnostic test with libraries of cell surface expressed microbial protein variants to analyze binding affinity providing a quantified numerical value for affinity. In certain embodiments, the protein variants are expressed on the exterior of cells containing intracellular nucleic acids with barcodes that correlate to specific amino acid variants sequences encoded and expressed by vectors or other nucleic acids. In certain embodiments, the libraries are used in methods to confirm the effectiveness or non-effectiveness of the test binding agent to identify circulating mutant microbial strains, e.g., coronaviral strains of SARS-CoV-2. In certain embodiments the microbial protein is a coronavirus nucleocapsid sequence or coronavirus spike protein sequence.

In certain embodiments, this disclosure relates to methods of screening a binding agent across a library of microbial protein variants and determining the binding affinity against each variant. In certain embodiments, this disclosure relates to a library of cell surface coronavirus nucleocapsid expression constructs comprising nucleic acids having barcode sequences and sequences encoding peptides, wherein the peptides comprise an N-terminal signal peptide sequence for translocation of the peptide across a cell membrane, a coronavirus nucleocapsid sequence, and a transmembrane domain sequence for insertion into a cell membrane C-terminal to the coronavirus nucleocapsid sequence; wherein each of the encoded peptides are unique coronavirus nucleocapsid sequences with only one or at least a single amino acid variant when compared to a base coronavirus nucleocapsid sequence; and wherein the encoded peptides with the amino acid variants are correlated to single unique nucleotide barcode sequences for each cell.

In certain embodiments, the base coronavirus nucleocapsid sequence is a consensus coronavirus sequence, variant of interest, or variant of concern.

In certain embodiments, the microbial protein variants are encoded by barcoded nucleic acids in operable combination with a heterologous promoter, internal ribosome entry site, or cap-independent translation element. In certain embodiments, nucleic acid barcodes are downstream of the nucleic acid sequence encoding the microbial protein variant.

In certain embodiments, this disclosure relates to a library of cells expressing coronavirus nucleocapsid variant expression constructs as disclosed herein. In certain embodiments, the encoded peptides of the library of coronavirus nucleocapsid expression constructs or other protein expression constructs contains variants at each position in the base coronavirus nucleocapsid sequence. In certain embodiments, the single amino acid variants are alanine, arginine, asparagine, aspartic acid, cysteine, glutamine, glutamic acid, glycine, histidine, isoleucine, leucine, lysine, phenylalanine, proline, serine, threonine, tryptophan, tyrosine, methionine, and valine at each position within the base coronavirus nucleocapsid sequence.

In certain embodiments, the encoded peptides further comprise a peptide marker/label sequence. In certain embodiments, the peptide marker/label sequence is between the signal peptide and the coronavirus nucleocapsid sequence.

In certain embodiments, this disclosure relates to computer readable medium comprising the data of each of the unique nucleotide barcode sequence that are associated with each cell that encode and express coronavirus nucleocapsid sequence variants.

In certain embodiments, this disclosure relates to methods of detecting a test binding agent for binding to coronavirus nucleocapsid variants comprising: contacting a test binding agent specific for a coronavirus nucleocapsid with a library of cells expressing coronavirus nucleocapsid variant expression constructs as reported herein providing cells with the test binding agent bound to coronavirus nucleocapsid variants on the cells; contacting the test binding agent bound to coronavirus nucleocapsid variant on the cells with a secondary labeling agent capable of binding the test binding agent bound to the coronavirus nucleocapsid variants on the cells providing secondary labeling agent bound to the coronavirus nucleocapsid variants on the cells.

In certain embodiments, methods further comprise separating/purifying/isolating cells with the secondary labeling agent bound to the coronavirus nucleocapsid variant e.g., by fluorescent activated cell sorting, and sequencing the nucleic acids inside the cells. In certain embodiments, methods further comprise sequencing the barcode of the separated/purified/isolated cells and associating the barcodes to a specific coronavirus nucleocapsid variant.

In certain embodiments, methods include associating the barcodes to a specific coronavirus nucleocapsid variant is by comparing the barcode sequence to sequences on computer readable medium comprising the data of each of the unique nucleotide barcode sequence that are associated with each of coronavirus nucleocapsid sequence variants in the library.

In certain embodiments, methods further comprise identifying the cells associated with the coronavirus nucleocapsid variants that are bound by the test agent or do not bind the test agent, and/or quantifying the binding affinity to the coronavirus nucleocapsid variants on the cells.

In certain embodiments, methods further comprise contacting the cells with a labeling agent capable of binding the peptide label sequence between the signal peptide and the coronavirus nucleocapsid sequence, and detecting the labeling agent, indicating that the test compound is bound to the coronavirus nucleocapsid sequence on the cells.

In certain embodiments, this disclosure relates to methods of screening a binding agent across a library of microbial protein variants and determining the binding affinity against each variant. In certain embodiments, this disclosure relates to a library of cell surface expressing coronavirus spike protein expression constructs comprising nucleic acids having barcode sequences and sequences encoding peptides, wherein the peptides comprise an N-terminal signal peptide sequence for translocation of the peptide across a cell membrane, a coronavirus spike protein sequence, and a transmembrane domain sequence for insertion into a cell membrane C-terminal to the coronavirus nucleocapsid sequence; wherein each of the encoded peptides are unique coronavirus spike protein sequences with only one or at least a single amino acid variant when compared to a base coronavirus spike protein sequence; and wherein the encoded peptides with the amino acid variants are correlated to single unique nucleotide barcode sequences for each cell.

In certain embodiments, this disclosure relates to a library of cells expressing coronavirus spike protein variant expression constructs as disclosed herein.

In certain embodiments, the encoded peptides of the library of coronavirus spike protein expression constructs contains variants at each position in the base coronavirus spike protein sequence. In certain embodiments, the single amino acid variants are alanine, arginine, asparagine, aspartic acid, cysteine, glutamine, glutamic acid, glycine, histidine, isoleucine, leucine, lysine, phenylalanine, proline, serine, threonine, tryptophan, tyrosine, methionine, and valine at each position within the base coronavirus spike protein sequence.

In certain embodiments, the encoded peptides further comprise a peptide marker/label sequence. In certain embodiments, the peptide marker/label sequence is between the signal peptide and the coronavirus spike protein sequence.

In certain embodiments, this disclosure relates to computer readable medium comprising data associated with each of the unique nucleotide barcode sequence that are associated with each cell that encode and express coronavirus spike protein sequence variants.

In certain embodiments, this disclosure relates to methods of detecting a test binding agent for binding to coronavirus spike protein variants comprising: contacting a test binding agent specific for a coronavirus spike protein with a library of cells expressing coronavirus spike protein variant expression constructs as reported herein providing cells with the test binding agent bound to coronavirus spike protein variants on the cells; contacting the test binding agent bound to coronavirus spike protein variant on the cells with a secondary labeling agent capable of binding the test binding agent bound to the coronavirus spike protein variants on the cells providing secondary labeling agent bound to the coronavirus spike protein variants on the cells.

In certain embodiments, methods further comprise separating/purifying/isolating cells with the secondary labeling agent bound to the coronavirus spike protein variant e.g., by fluorescent activated cell sorting.

In certain embodiments, methods further comprise sequencing the barcode of the separated/purified/isolated cells and associating the barcodes to a specific coronavirus spike protein variant.

In certain embodiments, associating the barcodes to a specific coronavirus spike protein variant is by comparing the barcode sequence to sequences on computer readable medium comprising the data of each of the unique nucleotide barcode sequence that are associated with each of coronavirus spike protein sequence variants in the library.

In certain embodiments, methods further comprise identifying the cells associated with the coronavirus spike protein variants that are bound by the test agent or do not bind the test agent, and/or quantifying the binding affinity to the coronavirus spike protein variants on the cells.

In certain embodiments, methods further comprise contacting the cells with a labeling agent capable of binding the peptide label sequence between the signal peptide and the coronavirus spike protein sequence, and detecting the labeling agent, indicating that the test compound is bound to the coronavirus spike protein sequence on the cells.

In certain embodiments, this disclosure relates to methods of screening a binding agent across a library of cells expressing single unique microbial protein variants and determining the binding affinity against each variant. In certain embodiments, the library of cells expressing a single variant is greater than 1000, 2000, 4000, or 7000 cells.

In certain embodiments, this disclosure relates to a library of cells expressing coronavirus nucleocapsid or other viral protein variant expression constructs as disclosed herein. In certain embodiments, this disclosure relates to a library of cell surface expressing coronavirus nucleocapsid expression constructs comprising nucleic acids having barcode sequences and sequences encoding peptides, wherein the peptides comprise an N-terminal signal peptide sequence for translocation of the peptide across a cell membrane, a coronavirus nucleocapsid sequence or other viral protein, and a transmembrane domain sequence for insertion into a cell membrane C-terminal to the coronavirus nucleocapsid sequence or other viral protein; wherein each of the encoded peptides are unique coronavirus nucleocapsid or other viral protein sequences with only one or at least a single amino acid variant when compared to a base coronavirus nucleocapsid sequence base viral protein sequence; and wherein the encoded peptides with the amino acid variants are correlated to single unique nucleotide barcode sequences for each cell.

In certain embodiments, the encoded peptides of the library of coronavirus nucleocapsid expression constructs contains variants for substantially all of the position in the base coronavirus nucleocapsid sequence except for methionine at position 1. In certain embodiments, the encoded peptides of the library of coronavirus nucleocapsid expression constructs contains variants for at least 400, 410, 415, 416, 417, or 418 continuous position in the base coronavirus nucleocapsid sequence. In certain embodiments, the single amino acid variants are alanine, arginine, asparagine, aspartic acid, cysteine, glutamine, glutamic acid, glycine, histidine, isoleucine, leucine, lysine, phenylalanine, proline, serine, threonine, tryptophan, tyrosine, methionine, and valine at each position within the base coronavirus nucleocapsid sequence. In certain embodiments, the encoded peptides further comprise a peptide marker/label sequence. In certain embodiments, the peptide marker/label sequence is between the signal peptide and the coronavirus nucleocapsid sequence or other protein variants.

In certain embodiments, this disclosure relates to computer readable medium comprising the data of each of the unique nucleotide barcode sequences, nucleic acid sequences, mutant positions, and/or protein sequences that are associated with each cell that encode and express coronavirus nucleocapsid sequence variants or other protein variants.

In certain embodiments, this disclosure relates to methods of detecting a test binding agent for binding to coronavirus nucleocapsid variants comprising: contacting a test binding agent specific for a coronavirus nucleocapsid with a library of cells expressing coronavirus nucleocapsid variant expression constructs as reported herein providing cells with the test binding agent bound to coronavirus nucleocapsid variants on the cells; contacting the test binding agent bound to coronavirus nucleocapsid variant on the cells with a secondary labeling agent capable of binding the test binding agent bound to the coronavirus nucleocapsid variants on the cells providing secondary labeling agent bound to the coronavirus nucleocapsid variants on the cells, and detecting the secondary labeling agent.

In certain embodiments, methods further comprise separating/purifying/isolating cells with the secondary labeling agent bound to the coronavirus nucleocapsid variant e.g., by fluorescent activated cell sorting. In certain embodiments, methods further comprise sequencing the barcode of the separated/purified/isolated cells and associating the barcodes to a specific coronavirus nucleocapsid variant. In certain embodiments, associating the barcodes to a specific coronavirus nucleocapsid variant is by comparing the barcode sequence to sequences on computer readable medium comprising the data of each of the unique nucleotide barcode sequence that are associated with each of coronavirus nucleocapsid sequence variants in the library.

In certain embodiments, methods further comprise identifying the cells associated with the coronavirus nucleocapsid variants that are bound by the test agent or do not bind the test agent, and/or quantifying the binding affinity to the coronavirus nucleocapsid variants on the cells.

In certain embodiments, methods further comprise contacting the cells with a labeling agent capable of binding the peptide label sequence between the signal peptide and the coronavirus nucleocapsid sequence, and detecting the labeling agent, indicating that the test compound is bound to the coronavirus nucleocapsid sequence on the cells.

### Deep Mutational Scanning Identifies SARS-CoV-2 Nucleocapsid Escape Mutations of Currently Available Rapid Antigen Tests

To evaluate the impact of mutations on antibodies used in commercially available antigen tests, antibody bindings were measured for all possible nucleocapsid point mutations using a mammalian surface-display platform and deep mutational scanning. The results provide a complete map of the epitopes of the antibodies and their susceptibility to mutational escape. The antibody escape mutational profiles generated here serve as a valuable resource for predicting the performance of rapid antigen tests.

Herein is reported a platform for mammalian surface-display of the SARS-CoV-2 nucleocapsid, an intracellular protein that allows for direct and quantitative measurement of antibody binding. This platform was combined with a site-saturated mutational scanning library containing all possible nucleocapsid protein, single amino acid substitutions along the entire nucleocapsid protein sequence. The approach measures the effect of all possible nucleocapsid protein mutations on antibody binding in a single experiment and generates a complete, unique escape mutational profile for each antibody. Escape mutational profiles are characterized by distinct regions of high and low escape scores that clearly identify both the epitopes and the vulnerabilities of diagnostic antibodies to mutations within and distal to the epitope.

The results show that rapid antigen tests are well-positioned to detect the mutations found in previous and current variants of concern. Furthermore, the data generated here contain binding measurements for all possible amino acid substitutions that may arise in future variants and thus are a valuable resource for the continued, accurate tracking of COVID-19 infections. The combination of mammalian surface-display with deep mutational scanning (DMS) is a generalizable method to study the effects of antigen mutations on antibody binding or protein-protein interactions more broadly in a suitable expression system.

Described herein are methods to evaluate how mutations in the main target of antigen tests, the SARS-CoV-2 nucleocapsid protein, affect recognition by diagnostic antibodies. The binding properties of antibodies from 11 commercial antigen tests were evaluated to all possible mutations in the nucleocapsid protein. For each antibody tested, the results provide a comprehensive list of antigen mutations with the potential to evade detection in the associated diagnostic test.

These data indicate that SARS-CoV-2 nucleocapsid mutations found in previous and current variants of concern and interest did not affect diagnostic test performance. Evaluation of the diagnostic tests with sequence-confirmed remnant clinical samples confirms this prediction.

Further, these mutational scanning data go beyond the mutations already present in sequence databases and may predict the performance of diagnostic antibodies against possible future mutations. When a new variant arises with novel nucleocapsid mutations, the data to predict test performance is already available. Thus, this study serves as a powerful resource with direct clinical and public health impact.

Atomic resolution structures of antibody-antigen complexes, currently the gold standard for epitope mapping, provide an accurate location of an antibody epitope, with detailed information about the atomic contacts between the two molecules. Structures cannot, however, determine how an individual mutation will affect the affinity of their interactions. Instead, they rely on predictions based on our knowledge of the physicochemical properties of the interacting amino acids. Other approaches use linear peptides that do not faithfully reflect 3D epitopes. Considering that 16 out of the 17 SARS-CoV-2 diagnostic antibodies investigated as part of this study target 3D epitopes, linear mapping is highly limited because short peptides may not cover enough of the epitope, are unlikely to assume the native fold, and will expose typically buried residues. These limitations are overcome by employing mammalian surface-display to directly probe antibody recognition of the full-length antigen and using a mutational library to generate binding measurements for all possible antigen mutations.

This approach does not directly determine an epitope of an antibody—the area of physical contact between antibody and antigen. Most escape mutations will be at the binding interface and reduce affinity through direct mechanisms, especially in the case of linear epitopes. Some mutations, however, will reduce binding indirectly through allostery. This is especially important for antibodies recognizing 3D epitopes, as mutations far away from the binding site may reduce the stability of the protein or affect local structure at the epitope allosterically. Thus, while escape mutations mapped onto the structure of the antigen identify localized surface patches, they represent a combination of the epitope and additional sites that are important for maintaining the conformational integrity and accessibility of the epitope. This approach thus goes beyond the notion of an epitope and, instead, provides a mutational profile reminiscent of a fingerprint of the antibody on the antigen. These fingerprints are highly valuable in the evaluation of antibodies and other detection reagents, such as nanobodies or DNA aptamers used in a diagnostic test, regarding their ability to detect variants of a rapidly mutating viral antigen.

Numerous examples of allosteric effects were uncovered on the antibody recognition of nucleocapsids. The antibodies 1C1 and Ab166, for example, are sensitive to mutations in R262, which is located at the back of the dimerization domain, distal to the epitopes. Several substitutions at R262, particularly changes to small non-polar amino acids (A, V, L, I, and M), do not affect antibody binding, suggesting that R262 supports the structural integrity of the epitope via its aliphatic side chain rather than making physical contact with the antibody. A264, located adjacent to R262, exhibits a similar escape profile for 1C1 and Ab166, consistent with a structural role and allosteric effects on antibody binding. Substitutions to charged and polar amino acids are most disrupting, while small hydrophobic side chains are tolerated for binding. In the N-RBD, antibody MM08 is highly sensitive to mutations at K143, which is not at the surface and at a significant distance from the epitope, defined by the most sensitive sites (L139, A138, and G137). K143 hydrogen bonds with the backbone oxygen of L139, a critical interaction maintaining the structural integrity of the epitope.

Methods disclosed here are the first to combine a mammalian surface-display platform with DMS. It combines the lentivirus-mediated stable mammalian surface-display of an intracellular protein with a barcoded, site-saturated mutational library, FACS, and high-throughput sequencing into a generalizable and efficient platform to comprehensively characterize not only antibody epitopes but also protein-protein interactions more broadly. Once the stable cell line expressing the mutational library is established, a screen can be performed from cells to library sequencing in 2 days, and multiple interactions can be mapped in parallel. It is contemplated that this platform is useful for evaluating various other questions surrounding the humoral response to pathogens. Further, this platform could be used to generate new insight into the process of affinity maturation in germinal center B cells. Mapping escape mutational profiles at progressive stages during the process could elucidate the interplay between gains in antibody affinity, specificity, and robustness toward antigen mutations.

### A Mammalian Surface-Display Platform for SARS-CoV-2 Nucleocapsid Protein

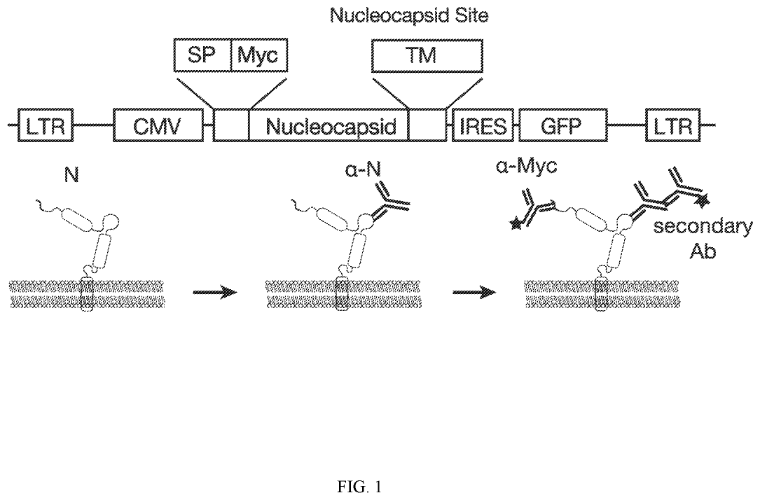

To display nucleocapsid protein on the surface of mammalian cells, an expression construct was generated containing nucleocapsid protein framed by an N-terminal signal peptide (SP) derived from IgG4 and a C-terminal transmembrane region (TM) derived from PDGFR. A Myc-tag was inserted between the SP and Nucleocapsid protein (N) and served to control for differences in expression levels. This construct was cloned into a lentiviral expression plasmid containing a 3′ internal ribosomal entry site (IRES) followed by GFP (Green Fluorescent Protein), which served as a selection marker (FIG. 1). To validate the surface-display system, a stable cell line expressing the Wuhan nucleocapsid protein was generated and tested for anti-N antibody binding. Cells were incubated with increasing concentrations of anti-N antibodies followed by staining with fluorescently labeled secondary and anti-Myc antibodies. Cells were then analyzed by flow cytometry, and anti-N antibody binding was determined from GFP+ and Myc+-gated cells. Dissociation constants measured were consistent with data collected using recombinant nucleocapsid protein and biolayer interferometry (BLI).

### A Deep Mutational Sequencing (DMS) Library of the Entire SARS-CoV-2 Nucleocapsid Protein Sequence

A site-saturated library was designed containing all amino acid substitutions of the Wuhan nucleocapsid protein (amino acids 2-419, no substitution a position 1 for methionine), using the same flanking regions for surface display. The library was amplified by PCR to introduce 15-nucleotide barcodes immediately downstream of the nucleocapsid protein coding sequence. Two replicate libraries were cloned into the pLVX-IRES-ZsGreen1 lentiviral expression vector and bottlenecked at approximately 150,000 barcoded constructs. PacBio™ long-read sequencing was employed to generate a lookup table associating unique barcodes with single amino acid mutants. More than 80% of reads with the correct sequence length contained a single mutation and mutations were marked by an average of 12.5 and 17.1 barcodes in libraries #1 and #2, respectively. Within the correct sequences with single mutations, libraries #1 and #2 contained 7,893 (99.4%) and 7,901 (99.5%) out of 7,942 possible mutations, respectively, of the entire nucleocapsid protein sequence. Mutations covered almost the entire sequence space, with only two sites (251 and 252) largely missing where the input library had failed to generate mutants.

The plasmid library was packaged into lentiviral particles, which were then used to transduce HEK293 cells at a multiplicity of infection of approximately 0.1 to ensure that the majority of cells expressed a single nucleocapsid protein mutant. Fluorescence-activated cell sorting (FACS) was used to isolate successfully transduced GFP+ cells, which were subsequently stained with a fluorescently labeled anti-Myc antibody to isolate Myc+ cells. At least 5 million cells were selected at each step to ensure appropriate library coverage. The final GFP+ Myc+ library contains cells expressing substantially all possible stably folding mutations of SARS-CoV-2 nucleocapsid protein on their surface.

### Generation of a Stable Cell Line for Nucleocapsid Surface-Display

A codon-optimized Wuhan SARS-CoV-2 Nucleocapsid sequence (UniProt ID: PODTC9) was generated with N-terminal signal peptide sequence derived from IgG4 (SEQ ID NO: 1), MEFGLSWVFLVALFRGVQC, followed by a 1×GGS linker, Myc-tag (SEQ ID NO: 2) EQKLISEEDL, and a 2×GGS linker, as well as a C-terminal 5×GGS linker and followed by a transmembrane helix derived from PDGFR (SEQ ID NO: 3) AVGQDTQEVIVVPHSLPFKVVVISAILALVVLTIISLIILIMLWQKKPR. This sequence was cloned into pLVX-IRES-ZsGreen1 (TakaraBio™) at the EcoRI and NotI sites using Gibson assembly (NEB 2× Gibson Assembly Master Mix™). The resulting construct was packaged into a lentivirus using the packaging vectors psPAX2 and pMD2G and the LV-MAX™ lentiviral production system. Lentiviral titers were determined using the GFP reporter and a stable cell line was generated by infecting Viral Production Cells (from the LV-MAX™ lentiviral production system) at an MOI of 0.1 so that greater than 90% of cells are infected with a single viral particle. Cells were then sorted for GFP-expression using a FACS ARIA II™ instrument.

### Deep Mutational Surface-Display Library Generation

A site-saturation library containing all possible single amino acid mutations at positions 2-419 of the SARS-CoV-2 Nucleocapsid protein sequence (UniProt ID PODTC9) was synthesized. Fifteen-nucleotide barcodes were added by PCR using 5 amplification cycles using primers. The resulting DNA was assembled into pLVX-IRES-ZsGreen at the EcoRI and NotI sites. Assembly reaction was electroporated using Endura ElectroCompetent™ Cells, plated on LB+Ampicillin plates at an estimated 150,000 colonies per replicate library to limit library complexity, and grown overnight at 30° C. The next day cells were washed off the plates and plasmids were purified using the HiSpeed Plasmid Maxi™ kit. The resulting replicate libraries were packaged into a lentiviral library using the packaging vectors psPAX2 and pMD2G and the LV-MAX™ lentiviral production system. Lentivirus preparations were titered using the GFP reporter and stable cell lines were generated by infecting 200 million viral production cells at an MOI of 0.1 so that greater than 90% of cells are infected with a single viral particle. Cells were then sorted for GFP-expression to collect GFP-positive cells. GFP-positive cells were expanded and cells expressing functional nucleocapsid mutants were selected by sorting cells stained with a rabbit Alexa647-anti-Myc antibody at a 1:200 dilution. GFP-positive and Myc-positive cells were sorted and expanded for each replicate library. These cell lines were then used to screen against diagnostic antibodies.

### Library Sequencing and Analysis

Mutational plasmid libraries were digested using EcoRI and NotI to cut out inserts containing Nucleocapsid coding sequences and associated barcodes. Inserts were gel purified and sequenced. Circular consensus sequences (CSSs) were used to generate a lookup table containing unique barcodes and associated mutations. N protein sequences present in CSSs were identified by first identifying the constant regions at the 5′ (containing SP, Myc, and GGS linkers), followed by the constant 3′ regions (containing the TM helix and stop codon). The mutated region (N protein residues 2 to 419) was then translated, aligned with the Wuhan nucleocapsid reference sequence, and mutations were identified. Sequences with incorrect insert lengths or those not containing exactly one mutation were discarded. Approximately 3% of barcodes were mapped to multiple clones and were discarded as well. The resulting lookup table contained 7893 and 7901 unique mutations (99.4% and 99.5% of all possible N protein mutants) and 103,756 and 141,729 unique barcodes.

Fluorescence-Activated Cell Sorting of Libraries to Select Mutants that Escape Antibody Binding

Antibody escape mutations were identified using GFP-positive and Myc-positive stable cell libraries. For each antibody, 20 million cells were washed in PBS with 2.5% FBS and 10 mM HEPES (pH 7.5) and incubated for 30 minutes at room temperature with 1 mL of a concentration of antibody that will result in 90-95% saturation of surface-displayed Wuhan N. Cells were washed three times and then stained with appropriate host-specific anti-IgG and anti-Myc antibodies. Cells were sorted by FACS. Using the anti-Myc signal to account for differences in expression, diagonal gates were drawn on anti-Myc vs antibody signal plots to select the cells with the lowest 10-15% antibody signal. Between 3×10⁵ and 1.0×10⁶ cells were collected in production medium for each antibody and processed for identification of escape mutations.

### High-Throughput Sequencing of Sorted Cell Populations

Cells were washed once with PBS and RNA was extracted. cDNAs were prepared by reverse transcription using a specific primer designed to anneal immediately downstream of the barcode sequence. Barcodes were then amplified by 8 rounds of PCR using primers with a compatible overhang sequence. The purified amplicons were appended with dual-indexed bar codes. Libraries were validated by capillary electrophoresis, pooled, and sequenced achieve a depth of approximately 10 million reads per sorted sample and 50 million reads for each reference samples.

### Sequencing Data Analysis and Calculation of Escape Scores

Barcodes extracted from escape and reference population sequences were counted and the lookup table generated from sequencing results was used to identify the associated mutations. Counts of barcodes associated with the same mutation were summed to generate a total count for each mutation. Next, abundance scores were calculated for reference and escape populations as ni/N where ni is the count for mutation i and N is the sum of all counts in the respective population (N=Σini). To avoid artificially high escape scores, the lowest 5 percent of abundance values in the reference population were then set to the 5th-percentile value. Escape scores, Ei, were then calculated as the ratio of a mutation's abundance in the escape population and the abundance in the reference population (Ei=(ni,esc/Nesc/ni,ref/Nref)). To remove outliers the top 1% of escape score values were set to the 99th-percentile value.

The data were subjected to a series of transformations to generate a Z-normalized escape score. First, escape scores were normalized to values between 0 and 1, where xi are the scores before normalization and yi are the scores after normalization. Since the distributions resembled truncated normal distributions, an arcsine square root transformation was employed to produce a distribution more closely resembling a normal distribution. Z-normalization was performed to produce the final distribution with a mean of 0 and standard deviation of 1.

Identification of Nucleocapsid Mutations that Escape Diagnostic Antibody Binding

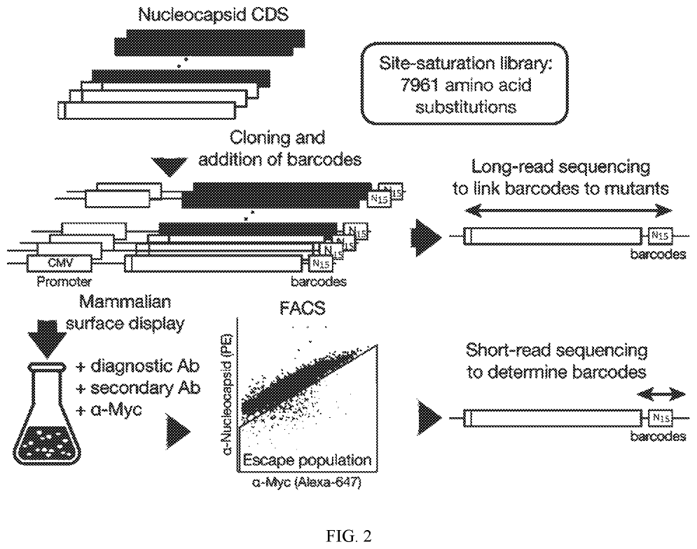

To test how nucleocapsid protein mutations affect recognition by diagnostic antibodies, flow cytometry combined with deep sequencing was used: diagnostic antibodies were bound to 20 million cells of each mutational library, and the escape population—cells with the lowest 10%-15% signal for antibody binding—were isolated by FACS (FIG. 2). Transcripts from cells in the escape population as well as the input library were subjected to deep sequencing and barcodes were counted in each sample to determine an escape score for the associated mutations. This score identifies the relative enrichment in the escape population and reflects the extent to which binding was reduced by a given mutation. A measurement of antibody binding for point mutations were obtained covering almost all of nucleocapsid protein mutational sequence space. Measurements between independent libraries and replicates using the same library generated similar escape mutational profiles. In the rest of the experiments, measurements were acquired with a single library.

Escape mutational profiles were determined for 17 monoclonal antibodies used in 11 SARS-CoV-2 rapid antigen tests. Escape mutational profiles of 3 antibodies were mapped onto the nucleocapsid protein sequence as a heatmap. Consistent with the small footprints of antibody binding sites, heatmaps reveal that a vast majority of mutations do not affect antibody binding while a small subset of mutations, clustered in well-defined sites, reduce binding considerably.

The linear epitope of R040 is a continuous stretch of amino acids located in a predicted disordered region outside of the folded domains and in which the majority of mutations strongly disrupt binding. Three-dimensional (3D) epitopes—such as those of C706 and 3C3—are characterized by discontinuous stretches in the primary sequence with varied degrees of escape. Many escape sites in 3D epitopes are single amino acids separated by stretches of amino acids in which mutations have virtually no effect on antibody recognition (FIG. 2).

Because all possible mutations are tested in this experiment, the data not only identify the sites of escape mutations but also how individual amino acid substitutions at these sites affect antibody recognition. 3C3, for instance, is sensitive to most substitutions at E323. At V324, however, only changes to charged or aromatic residues affect binding, while mutations to non-polar amino acids are recognized efficiently. Similarly, R040 binding is affected by all mutations at positions A397, D399, and D402. Yet, at position P396, R040 is sensitive to charged and polar amino acids but tolerates non-polar residues. Furthermore, mutations to residue L400, in the center of the epitope of R040, are mostly well-tolerated by the antibody, suggesting that it may bind to the amino acid backbone and not contact the side chain directly.

Together, these data show that mutations at distinct sites on the antigen affect antibody recognition and indicate the epitope location. Diverse profiles of escape mutations are identified and reveal that within individual sites some substitutions are tolerated less than others. These observations underscore the level of detail provided by this approach, which is not accessible to any other method currently available for epitope mapping.

### Validation of DMS Experiments

Of the 17 antibodies mapped, only a single antibody bound to a disordered region of the Nucleocapsid protein. R040 (SinoBiologicals™) is used in the Quidel Sofia™ SARS Antigen FIA Test, and a previous report showed that this test failed to detect specimens containing the mutation D399N, found in a small percentage of B.1.429 variants. Consistent with this report the epitope for R040 is confined to a continuous stretch of amino acids between residues L394 and F403, and that the mutation D399N is in the top 1% of escape scores for this antibody.

To further validate results from DMS experiments, a selection of individual mutations covering escape mutations from three antibodies (R040, 3C3, and C706) were cloned with different epitope locations and types (linear and 3D). Binding to point mutants was evaluated using antibody titrations on surface-displayed nucleocapsid protein. In agreement with the high-throughput screening results for each of the three antibodies, mutations within the epitopes reduced antibody binding, whereas mutations in other regions did not affect binding affinity.

All tested escape mutations for antibodies R040 (L395V, D399N, and D402V) and C706 (G85K, F110S, G116R, and R149D) abolished binding, whereas mutations outside the epitope had no effect. For 3C3, mutations E323V and V324E abolished binding and P326A reduced affinity by approximately 2 orders of magnitude. T329G reduced affinity only mildly but exhibited a decreased normalized overall antibody binding signal (normalized for expression by using the α-Myc signal). R040 and C706 antibodies, however, bind this mutant with a similar total signal as wild-type Wuhan. These observations suggest that T329G partially destabilizes the dimerization domain and, as a result, reduces the amount of actively folded protein available for binding to 3C3. The fraction of properly folded protein containing this mutation, however, binds with a similar affinity to the Wuhan sequence.

Like the effects of T329G, three of the four mutations within the N-RBD (G85K, F110S, and G116R) also reduced the total binding signal for 3C3 binding but had only mild effects on binding affinity. These sites are in the hydrophobic core of the N-RBD and likely unfold this domain (see section “epitopes in the RNA-binding domain”). This suggests that a denatured N-RBD may have long-range effects on epitope recognition in the dimerization domain. This may be due to indirect destabilization of the dimerization domain or occlusion of the epitope by unfolded peptide regions from the N-RBD. Consistent with this hypothesis, R149D, a mutation on the N-RBD surface, does not affect N-RBD stability and binds strongly to 3C3.

The high-throughput DMS experiment accurately identifies antibody escape mutations. Results from titration experiments with 3C3 and Nucleocapsid mutants further show that escape scores are affected by both reduced binding affinity and reduced availability of a conformational epitope.

### Epitopes in the Dimerization Domain

Five of the antibodies tested in this study bound to the Nucleocapsid's dimerization domain (N-DD, amino acids 257-364). The N-DD forms a symmetrical dimer in which two beta strands from each monomer form a central four-stranded antiparallel beta sheet with domain-swapped interactions. This core structure is surrounded at the back and on the sides by alpha helices of varying lengths. In contrast to the linear epitope of R040, escape mutations of antibodies binding to the N-DD are clustered in sets of sites that are discontinuous in the primary sequence. When mapped onto the structure of this domain, however, the escape sites cluster together in space, consistent with 3D epitopes.

Each antibody exhibited distinct escape mutational profiles across the N-DD. 2F4 is the only antibody binding to the alpha-helical backside of the domain. Escape mutations are clustered in residues P258, K261, A264, and K266 at the N terminus, as well as D297, P302, A305, and A311 in the central helical region. Mapped onto the surface of the protein, the escape sites highlight two adjacent patches in each monomer, indicating the epitope of the antibody.

The epitopes of 3C3, N-Ab3, and Ab166 are, in turn, located at the front face of the dimer with shared escape mutations in the loop connecting the two domain-swapped beta strands (residues T325 and P326). Their overall escape mutational profiles, however, differ markedly, which is consistent with distinct modes of antigen engagement. Escape mutations for 3C3 are located primarily within the loop (E323 to P326), while the epitope of Ab166 includes residues at the N-DD's N terminus (R262, A264, P279, E280, and T282), which are located adjacent to the beta strands and on the front face of the dimer. N-Ab3 escape mutations cover a larger section around the loop (M322 to W330) and further extend laterally toward the side distal to the dimerization interface, with highly sensitive sites on the C-terminal helix abutting the beta strands. Importantly, N-Ab3 escape mutations exclusively locate to one face of the helix (F346, K347, V350, 1351, and N354), whereas residues on the opposite face are not sensitive to mutations.

Consistent with the distinct binding modes of these three antibodies, mutations in loop residue T325 affect binding in different ways. 3C3 binding is affected by mutations to positively charged (K or R), but not negatively charged (D or E), amino acids. N-Ab3 exhibits the opposite behavior, while Ab166 is affected by both types of amino acids. These observations suggest that the antibody binding surface in contact with this residue contains positive charges in 3C3, is negatively charged in N-Ab3, and is possibly of a more hydrophobic character in Ab166.

Together, these observations highlight the rich information content generated by this mutational screening approach. Five antibodies were identified that bind to the same domain within the antigen. Although they have partially overlapped 3D epitopes, each antibody generates a distinct profile of escape mutations. These profiles not only set them apart from each other but also allow interpretations regarding the molecular mechanism causing reduced binding.

### Epitopes in the RNA-Binding Domain

The N-terminal of the RNA-binding domain of the Nucleocapsid protein (N-RBD, residues 47 to 174) contains a core of three antiparallel beta strands surrounded by long sections of ordered loops. An additional beta hairpin, which is critical for RNA-binding, protrudes from the core beta sheet. A total of ten antibodies in this study bind 3D epitopes in the N-terminal RNA-binding domain. Two distinct sets of escape sites were identified for these antibodies. The first set of sites is common to most or all antibodies, is enriched in non-polar and aromatic amino acids, is highly conserved, and maps to the compact hydrophobic core of the domain. Mutations at these sites most likely destabilize the domain, resulting in the unfolding of the 3D epitope and, consequently, reduced binding of the antibodies.

In addition to the destabilizing escape mutations, each antibody is characterized by distinct escape mutations in surface-accessible regions of the protein. When mapped onto the structure of the N-RBD, these sites are clustered in space, inferring the site of the antibody epitope. Based on this analysis, the epitopes on the N-RBD can be categorized into four main classes.

Class I epitopes were identified for MM08, C524, RC17604, and 1A7. These antibodies bind to loop regions between residues Y123 and K143 on the surface of the protein, opposite and distal to the beta hairpin with additional contributions from residues F66, R68, and G69. Loop regions contain the most sensitive sites for MM08, C524, and RC17604, while the main escape site of 1A7 is D81 on the surface side of a short helical segment surrounded by the loops.

MM05 defines class II epitopes. Its binding site is uniquely located in the loop of the beta hairpin at positions K95, D98, and M101. Additional less disruptive sites are found at positions P117, D128, and G129, which locate to a surface patch on the main body of the domain at the base of the beta hairpin. Of the N-RBD-binding antibodies, MM05 is least sensitive to mutations at the core sites. This is consistent with its epitope at the tip of the hairpin, the most distal epitope from the core structure, and suggests that the beta hairpin may form in the absence of a fully folded N-RBD.

Class III and class IV epitopes are characterized by escape mutations in distinct sites within the C-terminal loop regions (V158 to A173 for class III and R149 and P151 for class IV). C518 and R004 (class III) have allosteric contributions from core residues W108 and V133, and their epitopes are located on the surface of the domain's main body on the face opposite to class I epitopes. C706 and RC17602 (class IV) share their main escape mutations in the C-terminal loop and have highly similar overall escape mutational profiles. There are several clear differences, however, making each epitope unique. C706 has additional escape mutations in the C-terminal region around sites L161 and T166. RC17602, in turn, is sensitive to charged and polar substitutions at position A50 and has reduced sensitivity to core mutations at G71, V72, and P73.

These minor but distinguishing differences between two antibodies establish their unique fingerprints on the Nucleocapsid protein. The ability to distinguish even minor differences in antibody binding profiles highlights the power of the deep mutational scanning (DMS) method developed here. Similarly, it was identified that the antibodies mAb-1 and mAb-2, employed in the Clip COVID Rapid Antigen Test™, bind to the N-RBD and are equivalent to MM08 and R004. The escape mutational profiles of these antibodies are virtually identical and correlation between the data sets is high.

### Secondary Sites Affect Antibody Binding

Ab166 exhibited sites of elevated escape scores in a region outside of its epitope in the dimerization domain (residues G214 to D216). Specifically, mutations to small hydrophobic or aromatic amino acids reduced binding, whereas other mutations had mostly no effect. Titration experiments with individual mutations show that the total antibody binding signal is reduced, whereas affinity is unaffected. The most likely explanation is that hydrophobic, “sticky” residues may occlude the epitope on the dimerization domain in a fraction of molecules.

Secondary escape sites were also observed for a subset of antibodies (N-Ab3, Ab166, 1C1, 1A7, and R004) at the Nucleocapsid protein N-terminus in residues S2 to P6. A distinct, common pattern of escape mutations to small, non-polar residues in this part of the protein reduces binding to these antibodies, suggesting an identical mechanism of inhibition. Correlated with these is another set of sites with high escape scores in the region of R36 to R41. This region is part of a motif which forms a transient helix in molecular dynamics simulations, such that R32, R36, and R40 project in the same direction. This is, however, inconsistent with the pattern of escape mutations at R36, K38, R40, and R41 observed here. The positive charges at these sites appear to be required for efficient binding, suggesting charge-mediated interactions. The interactions may serve as a minor, secondary epitope binding to a negatively charged patch on some antibodies and contribute to enhanced affinity. As a result, loss of the positive charge and the resulting decreased antibody-antigen binding strength is detected in the mutational screen.

### Diagnostic Antibody Combinations Target Spatially Separated Epitopes

Rapid antigen tests generally utilize two or more antibodies, one immobilized on a solid support and the second antibody in a mobile phase. Binding of both antibodies is required for a signal to be generated. Hence, antibody combinations used in these tests have been carefully optimized.

Six of the tests evaluated use antibodies with epitopes in the same domain in the nucleocapsid protein. Different antibodies used in the same tests have their highest escape scores in mutually exclusive locations of the nucleocapsid protein. Minimal overlap is observed only in regions of lower total escape scores, which are likely allosteric sites outside the physical epitope.

When mapped onto the nucleocapsid protein structure, epitope locations of antibody combinations are in spatially separate locations, consistent with the ability of all antibodies to bind simultaneously. The GenBody™ COVID-19 Ag test uses antibodies 3C3 and 2F4, which bind to opposite faces of the dimerization domain, thus truly sandwiching the antigen. Negative stain electron microscopy, using a recombinant, purified SARS-CoV-2 Nucleocapsid dimerization domain with both antibodies, confirms this finding and shows the two antibodies facing each other with two Nucleocapsid dimers sandwiched between them.

Five products utilize multiple antibodies recognizing the N-RBD. The BD Veritor™ tests (BD Veritor At-Home COVID-19 Test™, and BD Verito System for Rapid Detection of SARS-CoV-2™) employ three different antibodies, all binding to the same domain. Each antibody in these tests recognizes a different epitope class. Together, these data show that DMS may be used to guide the selection of suitable antibody pairs in the design of new antigen tests.

Escape Mutational Profiles are Consistent with Laboratory Testing Results

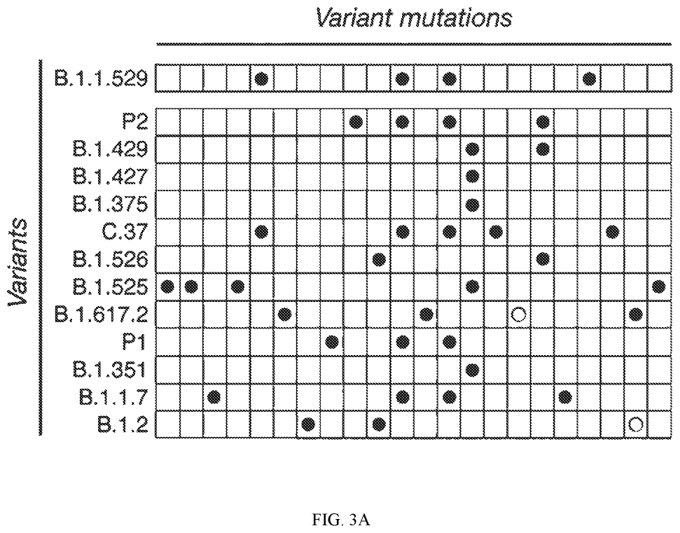

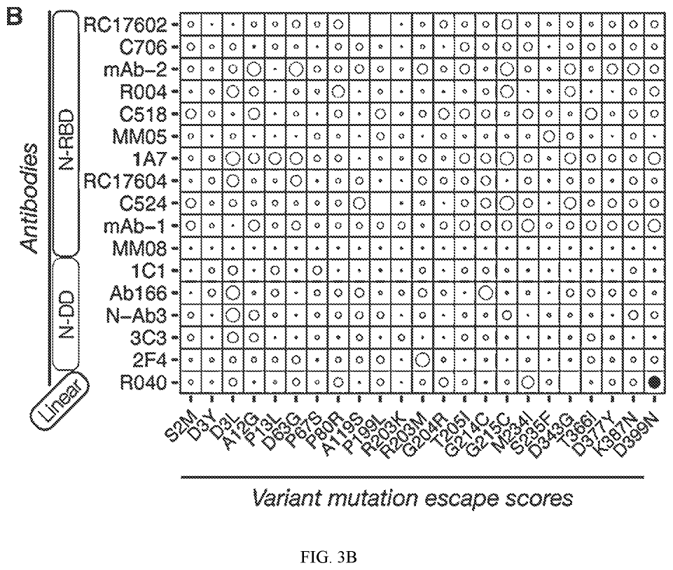

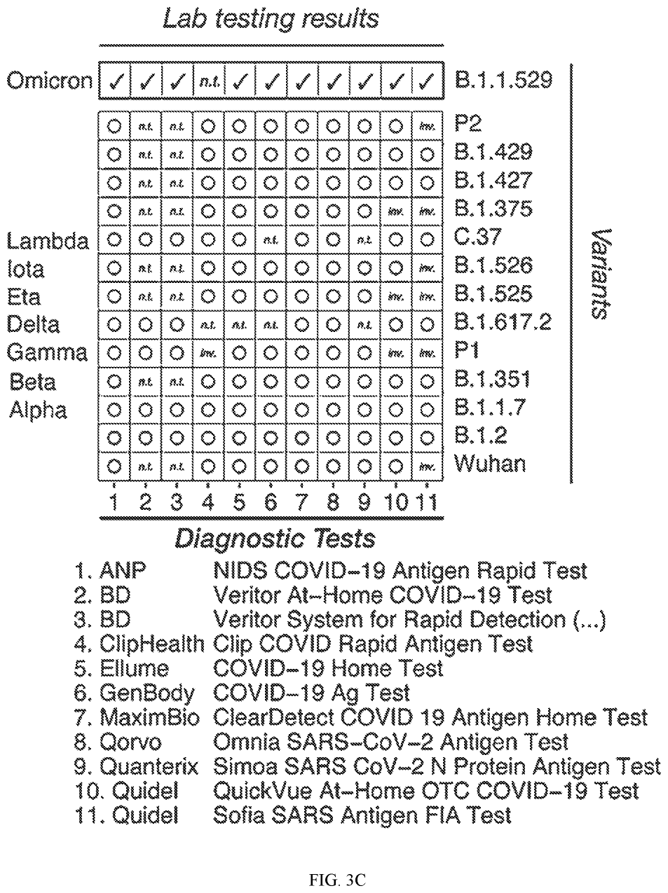

Deep mutational scanning data was used to predict antibody performance against mutations found within variants of concern and interest (FIG. 3A-3C). First, a weighted escape score, Ew, was calculated for each mutation so that Ew=Ei,j×Etotal,j, where Ei,j is the normalized escape score of mutation i at position j (0<Ei,j<1) and Etotal,j is the normalized total escape score at position j (0<Etotal,j<1; Etotal,j=(ΣjEi,j/max(ΣjEi,j))). This score considers the full range of mutational escape scores at each site and removes rare escape mutation outliers at sites with otherwise low individual escape scores.

Weighted escape scores predict full escape for the D399N mutation with antibody R040 used in the Quidel Sofia SARS-CoV-2 Antigen Test™, consistent with published lab test results (FIG. 3B). Almost all other mutations exhibited low weighted escape scores, suggesting that the variants containing these mutations do not affect the performance of diagnostic tests utilizing the antibodies evaluated here. The mutation D3L, present in the B.1.1.7 variant, is part of the secondary epitopes identified for some of the antibodies. Besides D399N, it is the only mutation with slightly elevated weighted escape scores for antibodies Ab166, 1C1, and 3C3, and may affect their test performance.

To test predictions from mutational scanning data, the limit of detection (LOD) of the relevant diagnostic tests were evaluated using serial dilutions of panels prepared from remnant clinical samples. Sequence-verified remnant samples of variants B.1.2, B.1.1.7 (alpha), B.1.351 (beta), P.1 (gamma), B.1.617.2 (delta), B.1.525 (cta), B.1.526 (iota), C.37 (lambda), B.1.375, B.1.427, B.1.429, P2, and B.1.1.529 (Omicron BA.1) were obtained from which pools of variants of concern (VOCs) were created (FIG. 3C). The LOD was defined as the lowest virus concentration (highest cycle threshold CT) that was detected 95% of the time. Consistent with escape mutational profiles, tests did not have significant dropouts of any variants relative to either Wuhan WA1 or B.1.2 reference samples.

Tests by Quidel (QuickVue At-Home OTC COVID-19 Test™ and SofiaSARS Antigen FIA Test™) and ClipHealth (Clip COVID Rapid Antigen Test™) commonly exhibited LODs with high viral concentrations, resulting in some samples not being detected in the current study (FIG. 3C). It is important to note that use of the tests in the laboratory setting is not representative of a clinical or at-home setting with fresh, unprocessed patient samples. Samples used here for testing the tests were pooled remnant clinical samples and were heat-inactivated or gamma-irradiated before use. The purpose of the testing experiments described here is to compare the ability of diagnostic tests to detect different variants as predicted by our high-throughput escape mutational profiling data.

Many variants of concern contain multiple mutations in the Nucleocapsid relative to the ancestral Wuhan strain. Because our DMS library contained only single mutations, the data cannot accurately predict escape mutants arising from multiple point mutations with synergistic effects. However, it is contemplated that this method could be extended to the inclusion of multiple mutations. Differences in relative binding strengths can be determined by collecting data from multiple titration points that can be used to generate full binding curves. SARS-CoV-2 Nucleocapsid protein contains several potential posttranslational modifications (PTMs) including phosphorylations and glycolysations. PTMs may interfere with or—if an antibody recognizes a modified site—may be required for antibody binding. Although this screen does not evaluate PTMs, the use of mammalian cells ensures that PTMs are as close to physiological as possible. Generating additional libraries base on mutations from more current variants of interest or variants of concern is contemplated.

1. A library of coronavirus nucleocapsid expression constructs comprising:
nucleic acids having barcode sequences and segments encoding peptides;
the peptides comprising
an N-terminal signal peptide sequence for translocation of the peptides across a cell membrane;
a coronavirus nucleocapsid sequence; and
a transmembrane domain sequence for insertion into a cell membrane C-terminal to the coronavirus nucleocapsid sequence;
wherein each of the encoded peptides are unique coronavirus nucleocapsid sequences with at least a single amino acid variant when compared to a base coronavirus nucleocapsid sequence; and
wherein the encoded peptides with the amino acid variant sequences are correlated to single unique nucleotide barcode sequences.

2. The library of coronavirus nucleocapsid expression constructs of claim 1, wherein the encoded peptides include variants at each position in the base coronavirus nucleocapsid sequence.

3. The library of coronavirus nucleocapsid expression constructs of claim 2, wherein the single amino acid variants are alanine, arginine, asparagine, aspartic acid, cysteine, glutamine, glutamic acid, glycine, histidine, isoleucine, leucine, lysine, phenylalanine, proline, serine, threonine, tryptophan, tyrosine, methionine, and valine at each position within the base coronavirus nucleocapsid sequence.

4. The library of coronavirus nucleocapsid expression constructs of claim 1, wherein a peptide label sequence is between the signal peptide and the coronavirus nucleocapsid sequence.

5. The library of coronavirus nucleocapsid expression constructs of claim 4, wherein the peptide label sequence comprises (SEQ ID NO: 2) EQKLISEEDL.

6. The library of coronavirus nucleocapsid expression constructs of claim 1, wherein the nucleic acids encoding the coronavirus nucleocapsid sequence comprises (SEQ ID NO: 4)
TCTGATAACGGACCCCAAAATCAGCGCAACGCCCCGCGGATCACTTTCGGCG GACCAAGCGACAGCACCGGGTCTAACCAGAATGGCGAGAGGAGCGGGGCTCGCAG CAAACAGCGTAGACCCCAAGGACTGCCAAATAACACCGCTAGTTGGTTCACAGCCT TGACCCAGCACGGAAAGGAGGATCTGAAATTCCCAAGAGGGCAGGGCGTCCCAATC AACACTAATAGTTCTCCTGATGACCAGATAGGTTATTACAGGAGGGCCACCCGGCG GATCCGCGGCGGGGATGGAAAGATGAAAGATTTAAGTCCACGATGGTATTTTTACT ATCTGGGAACTGGCCCTGAAGCAGGGTTGCCGTACGGGGCTAACAAGGACGGTATT ATTTGGGTTGCTACAGAAGGGGCGCTGAATACTCCTAAAGACCACATTGGGACCCGT AATCCTGCTAATAACGCCGCCATCGTGTTGCAGCTGCCTCAAGGCACTACCCTTCCC AAAGGATTCTATGCTGAGGGTTCACGGGGCGGCTCTCAGGCATCCTCAAGGTCCAGT AGCAGATCAAGGAACAGCTCCCGCAATTCCACTCCAGGCTCATCTAGGGGTACTAG CCCTGCACGGATGGCTGGGAATGGGGGGGATGCAGCACTGGCGCTCTTATTGCTCG ATCGCCTCAATCAGTTAGAGTCAAAGATGAGTGGGAAAGGACAGCAGCAGCAGGGG CAGACGGTGACCAAAAAGTCCGCAGCTGAGGCGAGCAAGAAACCCAGGCAGAAGC GGACAGCGACCAAGGCTTACAATGTGACCCAAGCCTTCGGACGGCGCGGTCCAGAG CAGACCCAGGGCAACTTCGGGGATCAGGAGCTTATTCGTCAAGGCACTGATTATAA GCACTGGCCCCAGATCGCACAGTTTGCCCCCAGTGCCTCCGCTTTTTTTGGGATGAG CAGAATTGGCATGGAGGTGACACCTTCAGGCACGTGGCTCACATACACCGGGGCAA TTAAGCTGGATGATAAGGATCCCAATTTCAAGGACCAGGTCATTCTTCTGAACAAGC ACATTGATGCCTACAAGACATTCCCACCCACCGAGCCCAAGAAGGACAAGAAGAAG AAAGCAGATGAGACACAAGCCCTTCCACAACGGCAGAAAAAACAACAGACTGTTAC GCTGCTGCCCGCTGCCGACCTGGACGACTTTTCTAAGCAATTGCAGCAGAGTATGTC ATCCGCAGACTCAACTCAAGCC wherein a codon variant results in a single amino acid change.

7. The library of coronavirus nucleocapsid expression constructs of claim 6, wherein the codon variant for alanine is GCC, the codon variant for cysteine is TGC, the codon variant for aspartic acid is GAC, the codon variant for glutamic acid is GAG, the codon variant for phenylalanine is TTC, the codon variant for glycine is GGC, the codon variant for histidine is CAC, the codon variant for isoleucine is ATC, the codon variant for lysine is AAG, the codon variant for leucine is CTG, the codon variant for methionine of is ATG, the codon variant for asparagine of is AAC, the codon variant for proline of is CCC, the codon variant for glutamine is CAG, the codon variant for arginine is AGA, the codon variant for threonine is ACC, the codon variant for valine is GTG, the codon variant for tryptophan is TGG, and the codon variant for tyrosine is TAC.

8. The library of coronavirus nucleocapsid expression constructs of claim 1, wherein the N-terminal signal peptide sequence for translocation of the peptides across a cell membrane comprises (SEQ ID NO: 1) MEFGLSWVFLVALFRGVQC.

9. The library of coronavirus nucleocapsid expression constructs of claim 1, wherein the transmembrane domain sequence for insertion into a cell membrane comprises (SEQ ID NO: 3) AVGQDTQEVIVVPHSLPFKVVVISAILALVVLTIISLIILIMLWQKKPR.

10. A library of cells expressing coronavirus nucleocapsid variant expression constructs as in claim 1.

11. A non-transitory computer readable medium comprising the data of each of the unique nucleotide barcode sequences that are associated with each coronavirus nucleocapsid sequence variants of claim 1.

12. A method of detecting a test binding agent for binding to coronavirus nucleocapsid variants comprising:
contacting a test binding agent specific for a coronavirus nucleocapsid with a library of cells expressing coronavirus nucleocapsid variant expression constructs as in claim 10 providing cells with the test binding agent bound to coronavirus nucleocapsid variants on the cells;
contacting the test binding agent bound to coronavirus nucleocapsid variant on the cells with a secondary labeling agent capable of binding the test binding agent bound to the coronavirus nucleocapsid variants on the cells providing secondary labeling agent bound to the coronavirus nucleocapsid variants on the cells.

13. The method of claim 12 further comprising separating cells with the secondary labeling agent bound to the coronavirus nucleocapsid variant.

14. The method of claim 12 further comprising sequencing the barcode of the separated cells and associating the barcodes to a specific coronavirus nucleocapsid variant.

15. The method of claim 14 wherein associating the barcodes to a specific coronavirus nucleocapsid variant is by comparing the barcode sequence to sequences on computer readable medium comprising the data of each of the unique nucleotide barcode sequence that are associated with each of coronavirus nucleocapsid sequence variants in the library.

16. The method of claim 12 further comprising quantifying the binding affinity of the test binding agent associated to the coronavirus nucleocapsid variants on the cells.

17. The method of claim 16, further comprising contacting the cells with a labeling agent capable of binding a peptide label sequence between the signal peptide and the coronavirus nucleocapsid sequence and detecting the labeling agent indicating that the test compound is bound to the coronavirus nucleocapsid sequence.
